(19) United States
(12) Patent Application Publication
(10) Pub. No.: US 2014/0066527 A1
(43) Pub. Date: Mar. 6, 2014

(54) Synthesis Gas Reaction and Processing System

(71) Applicants: Melissa Gaucher, Quebec (CA); Louis Denomme, Quebec (CA); Gregory Carr, Humble, TX (US)

(72) Inventors: Melissa Gaucher, Quebec (CA); Louis Denomme, Quebec (CA); Gregory Carr, Humble, TX (US)

(21) Appl. No.: 13/772,782

(22) Filed: Feb. 21, 2013

Related U.S. Application Data
(60) Provisional application No. 61/611,888, filed on Mar. 16, 2012.

(51) Int. Cl. C07C 29/151

(52) U.S. Cl. CPC C07C 29/1516
USPC 518/705; 568/884; 252/373

(57) ABSTRACT

A process wherein synthesis gas is reacted to produce desired products, such as alcohols, and wherein by-products, such as methane, are reformed to provide hydrogen and carbon monoxide that is recycled to the feed of synthesis gas. The process also may provide for the recycle of unreacted hydrogen and unreacted carbon monoxide to the feed of synthesis gas.

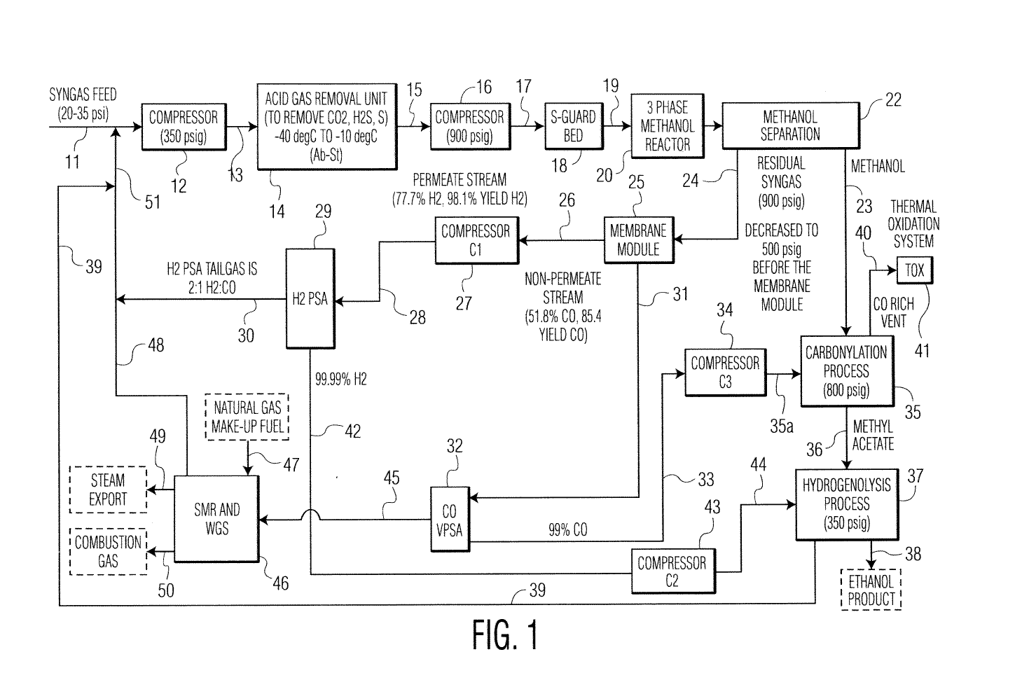

This application claims priority based on provisional Application Ser. No. 61/611,888, filed Mar. 26, 2012, the contents of which are incorporated by reference in their entirety.

This invention relates to a system for producing desired products, such as alcohols, from synthesis gas, and to reforming synthesis gas by-products. More particularly, this invention relates to producing desired products, such as alcohols, such as, for example, methanol and ethanol, from synthesis gas, and to reforming synthesis gas by products, such as, for example, methane, as well as reforming methane-containing gases, such as make-up natural gas, to produce hydrogen and carbon monoxide.

Synthesis gas, which includes hydrogen and carbon monoxide, and may contain additional residual components such as carbon dioxide and methane, may be used to produce other desired materials, such as, for example, alcohols and acetates.

For example, U.S. Pat. No. 8,080,693 discloses reacting hydrogen and carbon monoxide from synthesis gas to produce methanol. The methanol then is reacted with carbon monoxide, which may be obtained from synthesis gas, to produce a product comprising at least 25 mole % methyl acetate. The methyl acetate then is reacted with hydrogen, which also may be obtained from synthesis gas, to produce ethanol.

The present invention is directed to reforming by products contained in synthesis gas streams, as well as reforming methane-containing gases, such as make-up natural gas, to produce hydrogen and carbon monoxide, and to recycling such hydrogen and carbon monoxide to a fresh feed stream of synthesis gas, which is reacted to produce desired products such as alcohols.

In accordance with an aspect of the present invention, there is provided a process which comprises providing a feed stream of synthesis gas. The feed stream comprises hydrogen, carbon monoxide, and methane. The carbon monoxide and hydrogen are reacted to produce methanol. Unreacted synthesis gas, which includes hydrogen, carbon monoxide, and methane, is separated from the methanol. The unreacted synthesis gas then is passed through a separation zone, to provide a hydrogen-rich gaseous stream and a carbon monoxide-rich gaseous stream. The carbon monoxide-rich gaseous stream further includes methane. The carbon monoxide-rich gaseous stream is purified to provide purified carbon monoxide and a methane-rich gas stream. The methane-rich gas stream then is subjected to steam reforming to provide carbon monoxide and hydrogen. The carbon monoxide and hydrogen, that is produced as a result of the steam reforming of the methane-rich gas stream, are combined with the feed stream of synthesis gas.

The feed stream of synthesis gas may be obtained by any of a variety of methods for producing synthesis gas that are known to those skilled in the art. In a non-limiting embodiment, the synthesis gas is obtained by gasifying carbonaceous materials such as polyethylene and polypropylene residues, rubber residues, and biomass such as biological treatment sludge, forest biomass, agricultural biomass, wood products such as telephone poles and railroad ties, and urban biomass, including, but not limited to, municipal solid waste, or MSW. Examples of the gasification of such carbonaceous materials are disclosed in PCT Application No. WO 00/69994, and U.S. Pat. No. 8,080,693, the contents of which are incorporated herein by reference. When urban biomass is employed, such urban biomass may be obtained from municipal solid waste following sorting, drying (biologically or thermally using low grade heat from the gasification process), and size reduction.

In a non-limiting embodiment, prior to reacting the carbon monoxide with the hydrogen to produce methanol, the synthesis gas is treated to remove impurities therefrom. For example, in a non-limiting embodiment, the synthesis gas may be treated with cold methanol (e.g., at a temperature of from about −70° C. to about −10° C.) in an acid gas removal unit to remove impurities such as carbon dioxide, hydrogen sulfide, arsenic-containing compounds, chlorine, carbonyl-containing compounds, and sulfur, and/or be contacted with adsorbent materials and/or catalytic materials to remove such impurities.

Subsequent to the removal of impurities from the synthesis gas, the hydrogen and carbon monoxide of the synthesis gas are reacted to produce methanol. In a non-limiting embodiment, the synthesis gas that is reacted has a H₂:CO molar ratio of from about 1:1 to about 3:1, and includes CO₂ in an amount that does not exceed 6 mole %, methane in an amount that does not exceed 15 mole %, and water vapor in an amount that does not exceed 5 mole %. In another non-limiting embodiment, the methanol synthesis is effected under conditions such that the carbon monoxide is converted to methanol at a rate of up to 50 mole %.

In another non-limiting embodiment, the synthesis gas hereinabove described is reacted in the presence of an appropriate methanol synthesis catalyst. Suitable methanol synthesis catalysts include, but are not limited to, Cu/ZnO/Al₂O₃ catalysts. In yet another non-limiting embodiment, the synthesis gas is reacted in a “three phase” reactor, in which the methanol synthesis catalyst, such as a Cu/ZnO/Al₂O₃ catalyst, is suspended in an inert oil, such as mineral oil or Drakeol.

After the hydrogen and carbon monoxide of the synthesis gas are reacted to produce methanol, the unreacted synthesis gas, which contains unreacted hydrogen, unreacted carbon monoxide, and methane, and may include other components, is separated from the methanol.

In a non-limiting embodiment, the methanol is removed from the residual synthesis gas in a series of knock-out drums. The methanol/synthesis gas mixture is fed to the first knock-out drum at a temperature of about 140° C. and a pressure of about 990 psi. The methanol exits the final knock-out drum at a temperature of about 35° C. and a pressure of about 980 psi. The residual synthesis gas also exits the final knock-out drum in the gaseous phase at a temperature of about 35° C. and a pressure of about 980 psi. The unreacted synthesis gas, after being separated from the methanol, then is passed through a separation zone to provide a hydrogen-rich gaseous stream and a carbon monoxide-rich gaseous stream. The carbon monoxide-rich gaseous stream also includes methane.

In a non-limiting embodiment, the unreacted synthesis gas is passed to a membrane separation zone having one or more membranes, whereby hydrogen permeates the membrane(s) to provide a hydrogen-rich stream. The carbon monoxide does not permeate the membrane(s), and forms a carbon monoxide-rich stream. Examples of membranes which may be employed include, but are not limited to, hollow fiber membranes such as hollow polymer fiber membranes, such as polycarbonate fiber membranes, polyimide fiber membranes, and polysulfone fiber membranes, and membranes such as PRISM™, POLYSEP™, and VAPORSEP™. In another non-limiting embodiment, the membrane separation system includes a plurality of hollow polycarbonate fiber membranes.

In a non-limiting embodiment, the hydrogen-rich gaseous stream is purified to provide purified hydrogen and a tail gas stream that includes residual hydrogen, residual carbon monoxide, and carbon dioxide. The tail gas stream then is passed to the feed stream of synthesis gas hereinabove described.

In a non-limiting embodiment, the hydrogen-rich gaseous stream is purified by passing the hydrogen-rich gaseous stream through a pressure swing adsorption system. In a non-limiting embodiment, the pressure swing adsorption system includes one or more adsorption vessels, that is (are) packed with one or more adsorbents, such as, for example, activated carbon and/or zeolite adsorbents. As the hydrogen rich gas passes through the pressure swing adsorption system, impurities such as carbon dioxide, carbon monoxide, methane, residual hydrogen, hydrocarbons, and water are adsorbed selectively and temporarily at an elevated pressure, while an essentially pure (e.g., 99.99%) hydrogen gas passes through the pressure swing adsorption system. The carbon dioxide, carbon monoxide, methane, residual hydrogen, hydrocarbons, and water that were adsorbed are desorbed at a reduced pressure and removed from the adsorbent to provide a tail gas including hydrogen, carbon monoxide, and carbon dioxide. The tail gas then is treated at an elevated pressure to remove carbon dioxide. The resulting tail gas, which includes hydrogen and carbon monoxide, then may be recycled back and combined with the feed stream of synthesis gas.

In a non-limiting embodiment, the carbon monoxide-rich gaseous stream formed in the membrane separation zone is purified by passing the carbon monoxide-rich gaseous stream through a vacuum pressure swing adsorption zone, or through a pressure swing adsorption zone plus a vacuum swing adsorption zone. In a non-limiting embodiment, the vacuum pressure swing adsorption zone includes one or more adsorber vessels that include an appropriate adsorbent, such as, for example, a zeolite adsorbent. In a non-limiting embodiment, as the carbon monoxide-rich gas is passed through the vacuum pressure swing adsorption zone, 90% or more, by volume, of the carbon monoxide is adsorbed onto the adsorbent, and methane, carbon dioxide, nitrogen and other gases pass through the adsorbent to provide a tail gas rich in methane. The adsorbent then is depressurized to release a high purity (e.g., about 96%) carbon monoxide product.

The methane-rich gas stream then is subjected to steam reforming to provide carbon monoxide and hydrogen, which is combined with the feed stream of synthesis gas. The steam reforming is conducted under conditions which effect conversion of the methane-rich gas to hydrogen and carbon monoxide. In general, the methane-rich gas is contacted with superheated steam in a steam reformer. In a non-limiting embodiment, a make-up natural gas stream also may be fed to the steam reformer to provide additional methane. In another non-limiting embodiment, the steam reforming is effected at a temperature of from about 750° C. to about 1000° C. In another non-limiting embodiment, the steam reforming is effected at a temperature of from about 800° C. to about 900° C. In yet another non-limiting embodiment, the steam reforming is effected at a temperature of from about 840° C. to about 880° C.

In a non-limiting embodiment, the steam reforming is effected in the presence of an appropriate steam reforming catalyst, such as, for example, a nickel-based catalyst.

As noted hereinabove, the hydrogen and carbon monoxide produced as a result of subjecting the methane-rich gas to steam reforming are combined with the feed stream of synthesis gas, which then is purified and reacted to provide methanol as hereinabove described.

In a non-limiting embodiment, the methanol, which was produced from the hydrogen and carbon monoxide as hereinabove described, is reacted with the purified carbon monoxide to produce at least one acetate. The at least one acetate then is reacted with the purified hydrogen to produce at least one alcohol.

In a non-limiting embodiment, the at least one acetate is methyl acetate.

In another non-limiting embodiment, the methanol and purified carbon monoxide are reacted at a molar ratio of methanol to carbon monoxide of from about 2 to about 10. In yet another non-limiting embodiment, the methanol and purified carbon monoxide are reacted at a molar ratio of methanol to carbon monoxide of from about 2 to about 5.

In another non-limiting embodiment, the methanol and purified carbon monoxide are reacted at a temperature of from about 100° C. to about 350° C. In another non-limiting embodiment, the methanol and purified carbon monoxide are reacted at a temperature of from about 120° C. to about 280° C. In yet another non-limiting embodiment, the methanol and purified carbon monoxide are reacted at a temperature of from about 150° C. to about 250° C. In a further non-limiting embodiment, the methanol and purified carbon monoxide are reacted at a temperature of from about 200° C. to about 250° C.

The methanol and purified carbon monoxide, in a non-limiting embodiment, are reacted in the presence of a suitable catalyst for converting methanol and carbon monoxide to methyl acetate. Examples of suitable catalysts are described in U.S. Pat. No. 8,080,693.

In a non-limiting embodiment, the methyl acetate then is reacted with the purified hydrogen to produce ethanol.

In a non-limiting embodiment, the methyl acetate is reacted with the purified hydrogen at a temperature of from about 150° C. to about 300° C. In another non-limiting embodiment, the methyl acetate and the purified hydrogen are reacted at a temperature of from about 170° C. to about 275° C. In yet another non-limiting embodiment, the methyl acetate and purified hydrogen are reacted at a temperature of from about 200° C. to about 250° C.

In a non-limiting embodiment, the methyl acetate and purified hydrogen are reacted at a molar ratio of hydrogen to methyl acetate of at least 3. In another non-limiting embodiment, the methyl acetate and purified hydrogen are reacted at a molar ratio of hydrogen to methyl acetate of from about 5 to about 10.

In a non-limiting embodiment, the methyl acetate and purified hydrogen are reacted in the presence of a suitable hydrogenation catalyst. Hydrogenation catalysts which may be employed include, but are not limited to, Cu/Cr₂O₃, Cu/ZnO/Al₂O₃, Cu/Al₂O₃, Cu/ZnO/carbon, and combinations of Cu/Zn/Fe and Cu/Zn/Fe/Co on appropriate catalyst supports.

Thus, in a non-limiting embodiment, the present invention provides a process for producing ethanol from synthesis gas, wherein one or more by-products contained in the synthesis gas can be reformed to provide hydrogen and carbon monoxide that is recycled to the feed stream of synthesis gas, and/or unreacted hydrogen and unreacted carbon monoxide are recycled to the feed stream of synthesis gas.

The present invention provides an efficient method of reforming synthesis gas by products to produce hydrogen and carbon monoxide. Thus, in accordance with another aspect of the present invention, there is provided a process which comprises providing a crude synthesis gas. The crude synthesis gas comprises hydrogen, carbon monoxide, and methane. The crude synthesis gas is passed through a separation zone, which may be constructed and operated as hereinabove described, to provide a hydrogen-rich gaseous steam and a carbon monoxide-rich gaseous stream. The carbon monoxide-rich gaseous stream further includes methane. The carbon monoxide-rich gaseous stream is purified to provide purified carbon monoxide and a methane-rich gas stream. Such purification may be effected as hereinabove described. The methane-rich gas stream is subjected to steam reforming, under conditions which may be as hereinabove described, to provide carbon monoxide and hydrogen. The carbon monoxide and hydrogen then are combined with the crude synthesis gas.

In a non-limiting embodiment, the hydrogen-rich gaseous stream is purified to provide purified hydrogen and a tail gas stream including residual hydrogen, residual carbon monoxide, and carbon dioxide, which then is combined with the crude synthesis gas.

In another non-limiting embodiment, make-up natural gas is combined with the methane-rich gas stream, and the make-up natural gas and the methane-rich gas stream are subjected to steam reforming to provide carbon monoxide and hydrogen, which then are combined with the crude synthesis gas.

### BRIEF DESCRIPTION OF THE DRAWING

The invention now will be described with respect to the drawing, wherein:

The drawing is a schematic of an embodiment of the process of the present invention:

Referring now to the drawing, a feed of synthesis gas, which includes hydrogen, carbon monoxide, methane, and other components, is maintained at a pressure of 5 to 60 psig in line 11. A recycle stream of hydrogen, carbon monoxide, and carbon dioxide from line 51 is passed to line 11 and mixed with the synthesis gas feed. The synthesis gas feed and the recycle hydrogen and carbon monoxide in line 11 are passed to compressor 12, which compresses the gas to a pressure of from about 250 psig to about 400 psig. The compressed synthesis gas is withdrawn from compressor 12 through line 13, and is passed to acid gas removal unit 14. In acid gas removal unit 14, which is operated at a temperature of from about −70° C. to about −10° C., impurities such as CO₂, H₂S, arsenic-containing compounds, chlorine, carbonyl-containing compounds, and sulfur are removed from the synthesis gas. The treated synthesis gas then is withdrawn from acid gas removal unit 14 through line 15, and is passed to compressor 16, which further compresses the gas to a pressure of about 900 psig. The compressed synthesis gas is withdrawn from compressor 16 through line 17, and passed to guard bed 18, which contains a sulfur-removing material, such as for example, a CuCO₃/ZnCO₃ sulfur-removing catalyst, wherein any remaining sulfur in the synthesis gas is removed.

The synthesis gas then is withdrawn from guard bed 18 through line 19 and is passed to methanol reactor 20, wherein the carbon monoxide and hydrogen of the synthesis gas are reacted to produce methanol.

In a non-limiting embodiment, the methanol reactor 20 is a “three-phase” reactor, which includes a methanol synthesis catalyst, such as, for example, a Cu/ZnO/Al₂O₃ catalyst, that is suspended or dispersed in an inert oil, such as Drakeol.

In a non-limiting embodiment, the synthesis gas that is fed to methanol reactor 20 has a H₂:CO molar ratio of from about 1:1 to about 3:1, and includes methane in an amount that does not exceed 15 mole %. The synthesis gas also may include carbon dioxide in an amount that does not exceed 6 mole %.

The methanol and unreacted synthesis gas are withdrawn from methanol reactor 20 through line 21 and passed to methanol separation zone 22. Methanol separation zone 22 contains a series of knock-out drums. The first knock-out drum in the series is operated at a temperature of 140° C. and a pressure of 990 psi. The last knock-out drum in the series is operated at a temperature of 35° C. and a pressure of 980 psi. Methanol is withdrawn from methanol separation zone 22 through line 23 and passed to carbonylation reactor 35, in which methanol will be reacted with carbon monoxide to produce methyl acetate. Unreacted synthesis gas, which includes unreacted carbon monoxide, unreacted hydrogen, and methane, is withdrawn from methanol separation zone 22 through line 24, at a pressure of about 900 psig. In line 24, the pressure of the unreacted synthesis gas is reduced from about 900 psig to about 500 psig, such as, for example, by the use of a pressure regulator and/or regulating valves (not shown), and the unreacted synthesis gas is passed to membrane separation zone 25. Membrane separation zone 25 includes a plurality of hollow polymer fiber membranes, such as polycarbonate fiber membranes arranged in parallel, whereby hydrogen-rich and carbon monoxide-rich streams are created by gas permeation separation. Hydrogen permeates the membranes rapidly while the carbon monoxide-rich stream, which also includes methane, remains as the non-permeate gas.

The hydrogen-rich gas, which has permeated the membranes of membrane separation zone 25, experiences a pressure drop down to 40 psig, and then is withdrawn from membrane separation zone 25 through line 26 and is passed to compressor 27. In general, the pressure of the hydrogen-rich gas in compressor 27 is raised to a pressure of about 250 psig. Compressor 27 also may include a set of dual coalescing filters and activated carbon that remove any entrained oil from the hydrogen-rich gas.

The compressed hydrogen-rich gas is withdrawn from compressor 27 through line 28 and is passed to pressure swing adsorption unit 29 includes a plurality of pressure swing adsorption vessels, each of which is packed with activated carbon and zeolite adsorbents. As the hydrogen-rich gas is passed through pressure swing adsorption unit 29, carbon dioxide, carbon monoxide, methane, hydrocarbons, residual hydrogen, and water are adsorbed selectively and temporarily at elevated pressure, while 99.99% pure hydrogen gas passes through the adsorber vessels. During operation, one of the adsorber vessels is producing purified hydrogen gas while the other vessels are in different stages of pressure equalization, desorption or recharge. Cycle times for the pressure swing adsorption vessels in the pressure swing adsorption unit 29 in general are about 10 minutes, but production of 99.99% pure hydrogen is continuous. The carbon monoxide, carbon dioxide, methane, hydrocarbon, residual hydrogen, and moisture captured gases that were adsorbed in pressure swing adsorption unit 29 are desorbed from the activated carbon and zeolite adsorbents at a pressure of 5 psig to ensure efficient removal. A tail gas, which includes hydrogen, carbon monoxide, and carbon dioxide, is combined with hydrogen and carbon monoxide from line 48, hydrogen from line 39, and passed to line 51. The combined stream of hydrogen, carbon monoxide, and carbon dioxide in line 51 then is combined with the feed of synthesis gas in line 11.

Hydrogen having a purity of 99.99% is withdrawn from pressure swing adsorption unit 29, at a pressure of from about 200 psig to about 225 psig, through line 42. The hydrogen in line 42 then is passed to compressor 43. In compressor 43, the pressure of the pure hydrogen gas is raised to about 530 psig. The compressed hydrogen gas may, if necessary, be filtered in order to remove any entrained oil. The pure hydrogen gas then is withdrawn from compressor 43 through line 44, and is passed to hydrogenolysis reactor 37, wherein the hydrogen is reacted with methyl acetate to produce ethanol.

The carbon monoxide-rich non-permeate gas in membrane separation zone 25 is withdrawn from membrane separation zone 25 through line 31, at a pressure of about 490 psig, and is passed to vacuum pressure swing adsorption zone 32. In vacuum pressure swing adsorption zone 32, the pressure of the carbon monoxide rich stream is reduced from about 490 psig to about 225 psig. Vacuum pressure swing adsorption zone 32 includes a plurality of adsorber vessels that contain copper chloride promoted zeolite adsorbents. In a non-limiting embodiment, one of the adsorber vessels adsorbs 90% or more, by volume, of the feedgas CO content onto the promoted zeolite material, and allows methane, carbon dioxide, nitrogen, and other gas constituents to pass through the adsorbent. At the same time, another adsorber vessel is undergoing a depressurization with a vacuum boost to release a high purity (e.g., about 96%) carbon monoxide product stream. The remaining vessels may be in different stages of pressure equalization and recharge. Cycle times between automatic vessel duty swings in vacuum pressure swing adsorption zone 32 typically are from about 15 minutes to about 20 minutes.

The pressure of the purified carbon monoxide gas is boosted to about 30 psig, and then is withdrawn from vacuum pressure swing adsorption zone 32 through line 33, and is passed to compressor 34. In compressor 34, the pressure of the purified carbon monoxide is increased to 550 psig. Compressor 34 also may include a dual coalescing filter and an activated carbon train to remove any entrained oil. The compressed carbon monoxide then is withdrawn from compressor 34 through line 35a, and is passed to carbonylation reactor 35, wherein the carbon monoxide is reacted with methanol from line 23 to produce methyl acetate.

A tail gas, which is rich in methane and carbon dioxide, and may include nitrogen and other gas components, is pressurized to about 200 psig, and then is withdrawn from vacuum pressure swing adsorption zone 32 through line 45, and is passed to a steam methane reforming, or SMR, unit 46. Prior to entering the SMR unit 46, the pressure of the tail gas is reduced to about 45 psig, such as, for example, by use of a pressure regulator and/or regulating valves (not shown). In the steam methane reforming unit 46, the tail gas is combined with superheated steam, and a make-up natural gas stream from line 47, and is heated to a temperature of from about 840° C. to about 880° C. The steam methane reforming unit 46 also includes an appropriate reforming catalyst, such as, for example, a nickel-based catalyst.

In steam methane reformer 46, methane is reacted with the steam to produce hydrogen and carbon monoxide according to the following equation:

CH₄+H₂O→CO+3H₂

Also taking place is the water gas shift reaction:

CO+H₂O→CO₂+H₂

Alternatively, the tail gas, which is rich in methane and carbon dioxide, is subjected to “dry reforming”, wherein the methane and carbon dioxide are reacted in the absence of water or steam, and in the presence of a catalyst, to produce carbon monoxide and hydrogen according to the following equation:

CH₄+CO₂→2CO+2H₂

In a non-limiting embodiment, the above reaction is carried out in the presence of a Cu/Ni/MgO/ZrO₂ catalyst.

When the methane is subjected to steam reforming in steam methane reforming unit 46 to produce hydrogen and carbon monoxide, the hydrogen and carbon monoxide are withdrawn from the steam methane reforming unit 46 through line 48.

Unreacted steam is withdrawn from line 49, and combustion gas, or off gas, which includes nitrogen, argon, carbon dioxide, and steam, is withdrawn through line 50. Efficient heat recovery is obtained from the hydrogen and carbon monoxide in line 48 and the combustion gas in line 50. This heat may be used to generate all of the required superheated steam, as well as preheat the tail gas from line 45, and make-up fuel in line 47. The hydrogen and carbon monoxide in line 48 are combined with the tail gas from line 30, and hydrogen from line 39, to form a combined stream of hydrogen, carbon monoxide and carbon dioxide, in line 51, which is passed to line 11, where the hydrogen, carbon monoxide, and carbon dioxide are combined with a fresh feed of synthesis gas.

As noted hereinabove, methanol is withdrawn from methanol separation zone 22 through line 23, and is passed to carbonylation reactor 35, in which the methanol is reacted with carbon monoxide from line 35a to produce methyl acetate. Methyl acetate is withdrawn from carbonylation reactor 35 through line 36, while unreacted carbon monoxide is withdrawn from carbonylation zone 35 through line 40, and passed to a thermal oxidation, or TOX, zone 41, wherein excess gas is burned off to prevent the build-up of impurities. The methyl acetate in line 36 is passed to hydrogenation reactor 37, wherein the methyl acetate is reacted with hydrogen from line 44 to produce ethanol. Ethanol is recovered from hydrogenation reactor 37 through line 38, and unreacted hydrogen is withdrawn from hydrogenation reactor 37 through line 39. The unreacted hydrogen in line 39 may be combined with the hydrogen and carbon monoxide from line 48, and the tail gas from line 30, to form a combined stream of hydrogen, carbon monoxide, and carbon dioxide in line 51, which is passed to line 11 to be combined with a fresh feed of synthesis gas.

The disclosures of all patents and publications (including published patent applications) are incorporated herein by reference to the same extent as if each patent and publication were incorporated individually by reference.

It is to be understood, however, that the scope of the present invention is not to be limited to the specific embodiments described above. The invention may be practiced other than as particularly described and still be within the scope of the accompanying claims.

What is claimed is:

1. A process, comprising:
(a) providing a feed stream of synthesis gas, wherein said feed stream of synthesis gas comprises hydrogen, carbon monoxide, and methane;
(b) reacting said carbon monoxide with said hydrogen to produce methanol;
(c) separating unreacted synthesis gas from said methanol;
(d) passing said unreacted synthesis gas through a separation zone, to provide a hydrogen-rich gaseous stream and a carbon monoxide-rich gaseous stream, wherein said carbon monoxide-rich gaseous stream further includes methane;
(e) purifying said carbon monoxide-rich gaseous stream to provide purified carbon monoxide and a methane-rich gas stream;
(f) subjecting said methane-rich gas stream to steam reforming to provide carbon monoxide and hydrogen; and
(g) combining said carbon monoxide and said hydrogen from step (f) with said feed stream of synthesis gas in step (a).

2. The process of claim 1, and further comprising:
(i) purifying said hydrogen-rich gaseous stream of step (d) to provide purified hydrogen and a tail gas stream including residual hydrogen, residual carbon monoxide, and carbon dioxide; and)
combining said residual hydrogen, residual carbon monoxide, and carbon dioxide from step (i) with said feed stream of synthesis gas in step (a).

3. The process of claim 2, and further comprising:
(i) reacting said methanol produced in step (b) with said purified carbon monoxide produced in step (e) to produce at least one acetate; and
(ii) reacting said purified hydrogen with said at least one acetate to produce at least one alcohol.

4. The process of claim 3 wherein said at least one acetate is methyl acetate.

5. The process of claim 4 wherein said at least one alcohol is ethanol.

6. The process of claim 1 wherein, in step (f), make-up natural gas is combined with said methane-rich gas stream, and said make-up natural gas and said methane is subjected to steam reforming to provide carbon monoxide and hydrogen.

7. A process, comprising:
(a) providing a crude synthesis gas, wherein said crude synthesis gas comprises hydrogen, carbon monoxide, and methane;
(b) passing said crude synthesis gas through a separation zone, to provide a hydrogen-rich gaseous stream and a carbon monoxide-rich gaseous stream, wherein said carbon monoxide-rich gaseous stream further includes methane;
(c) purifying said carbon monoxide-rich gaseous stream to provide purified carbon monoxide and a methane-rich gas stream;
(d) subjecting said methane-rich gas stream to steam reforming to provide carbon monoxide and hydrogen; and
(e) combining said carbon monoxide and said hydrogen from step (d) with said crude synthesis gas in step (a).

8. The process of claim 7, and further comprising:
(i) purifying said hydrogen-rich gaseous stream of step (b) to provide purified hydrogen and a tail gas stream including residual hydrogen, residual carbon monoxide, and carbon dioxide; and
(ii) combining said residual hydrogen, residual carbon monoxide, and carbon dioxide from step (i) with said crude synthesis gas in step (a).

9. the process of claim 7 wherein, in step (d), make-up natural gas is combined with said methane-rich gas stream, and said make-up natural gas and said methane-rich gas stream are subjected to steam reforming to provide carbon monoxide and hydrogen.
